(19) United States
(12) Patent Application Publication
(10) Pub. No.: US 2025/0248410 A1
(43) Pub. Date: Aug. 7, 2025

(54) Method for Preparing Steamed Flour-based Products by Using A Thermostable Glucoamylase

(71) Applicant: NOVOZYMES A/S, Bagsvaerd (DK)

(72) Inventors: Qing XU, Beijing (CN); Yazhen WANG, Beijing (CN); Liyan HUANG, Beijing (CN)

(73) Assignee: NOVOZYMES A/S, Bagsvaerd (DK)

(21) Appl. No.: 18/856,644

(22) PCT Filed: Nov. 30, 2022
(86) PCT No.: PCT/EP2022/083874

§ 371 Date: Oct. 14, 2024

(30) Foreign Application Priority Data
Apr. 24, 2022 (WO) PCT/CN2022/088680

(51) Int. Cl. A21D 8/04; A21D 8/06

(52) U.S. Cl. CPC A21D 8/042; A21D 8/06; C12Y 302/01003

(57) ABSTRACT

The invention relates to methods of producing a steamed flour-based product, said methods comprise: providing a dough or a paste comprising flour and a thermostable glucoamy lase at least 70% identical to SEQ ID NO:1. SEQ ID 1×10;6. SEQ ID NO:7 or SEQ ID NO:8; and steaming the dough or the paste to produce a streamed flour-based product, as well as compositions comprising said thermostable glucoamylase.

### REFERENCE TO SEQUENCE LISTING

This application contains a Sequence Listing in computer readable form, which is incorporated herein by reference.

### FIELD OF THE INVENTION

The invention relates to an enzymatic method for preparing steamed flour-based product, in particular to a method for preparing steamed flour-based products such as steamed bread with a thermostable glucoamylase.

### BACKGROUND OF THE INVENTION

Steamed flour-based products such as steamed bread are one of the traditional staple foods of Chinese people, especially in the Northern China. In recent years, with the development of industrialization of food industry, the industrialized production of steamed flour-based products increased gradually.

WO2011/039324 disclosed a method for preparing a steamed bread, comprising the step of making a dough used to prepare steamed bread with one or more maltogenic alpha-amylases, one or more raw starch degrading enzymes, and at least one lipolytic enzyme, wherein the enzymatic method retards the staling of steamed bread products.

In daily life people often buy/make many steamed breads at one time and eat them in several times. The prepared steamed breads are stored at room temperature, cold temperature or frozen for a period of time, before eating again it is often necessary to re-steam the steamed bread in order to restore the softness and mouth feel. However, re-steaming negatively impacts the crumb structure, appearance color, chewiness and/or elasticity of the steamed flour-based products.

Thus, although some advancement in extension of shelf-life of steamed bread have been made using enzymatic treatments. However, there is still a need to provide new enzymatic solutions for improved performance in freshkeeping or anti-staling of steamed flour-based products, even if such steamed flour-based products need to be re-steamed.

### SUMMARY OF THE INVENTION

The inventors found that thermostable glucoamylases of the present invention showed greatly improved performance in freshkeeping or anti-staling of steamed flour-based products such as steamed bread, which were prepared by step of steaming the dough.

Another improved performance of the thermostable glucoamylases of the present invention was that they surprisingly and obviously improved the quality of the steamed flour-based products after re-steaming, such as improved appearance whiteness, softness, elasticity, crumb structure, and/or cohesiveness of the re-steamed products.

Another improved performance of the thermostable glucoamylases of the present invention was that they increased the sweetness or sweet taste of the product, the nature sweet is a preferred taste, which also allowed a reduction in the amount of added sugar in traditional recipes.

Accordingly, in a first aspect, a method of producing a steamed flour-based product, comprising:
a) providing a dough or a paste comprising flour and a glucoamylase at least 70% identical to SEQ ID NO: 1, SEQ ID NO:6, SEQ ID NO:7 or SEQ ID NO:8; and
b) steaming the dough or the paste to make the steamed flour-based product.

In a preferred embodiment of the present invention, the said glucoamylase is a mature thermostable variant of a parent glucoamylase.

A second aspect of the invention relates to dough premix or paste premix comprising a dough or a paste, and a mature thermostable variant of a parent glucoamylase as defined in the present invention.

In a preferred embodiment of the present invention, compared with the same conditions as other conditions but without adding the enzyme to the dough/the paste, the steamed flour-based products of the invention have reduced hardness and/or improved elasticity, and thus have an improved storage stability.

In a preferred embodiment of the present invention, compared with the same conditions as other conditions but without adding the enzyme to the dough/the paste, the steamed flour-based product of the present invention has an improved sensory evaluation after being stored at room temperature or low temperature, preferably, for a period of time.

In a preferred embodiment of the present invention, compared with the same conditions as other conditions but without adding the enzyme to the dough/the paste, the re-steamed steamed flour-based products have an improved sensory evaluation.

In a preferred embodiment of the present invention, the sensory evaluation is a comprehensive evaluation preferably, the average value, of softness, elasticity, surface whiteness, crumb structure, moisture, cohesiveness, chewiness and/or sweetness the steamed flour-based products.

Preferably, the glucoamylase of the invention is at least 71% identical to SEQ ID NO:1, SEQ ID NO:6, SEQ ID NO:7 or SEQ ID NO:8, e.g. at least 72%, e.g. at least 73%, e.g. at least 74%, e.g. at least 75%, e.g. at least 76%, e.g. at least 77%, e.g. at least 78%, e.g. at least 79%, e.g., at least 80%, e.g. at least 81%, e.g. at least 82%, e.g. at least 83%, e.g. at least 84%, e.g., at least 85%, e.g. at least 86%, e.g. at least 87%, e.g. at least 88%, e.g. at least 89%, e.g., at least 90%, e.g., at least 91%, e.g., at least 92%, e.g., at least 93%, e.g., at least 94%, e.g., at least 95%, e.g. at least 96%, e.g., at least 97%, e.g., at least 98%, e.g., at least 99% identical to SEQ ID NO: 1, SEQ ID NO:6, SEQ ID NO:7 or SEQ ID NO:8, more preferably, the glucoamylase is a mature thermostable variant of a parent glucoamylase.

### FIGURES

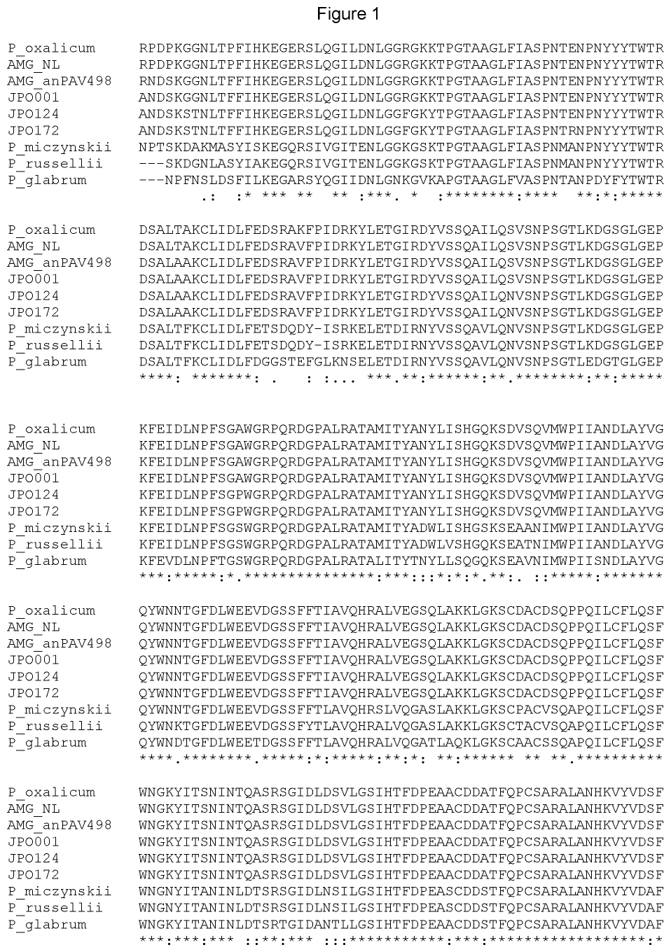

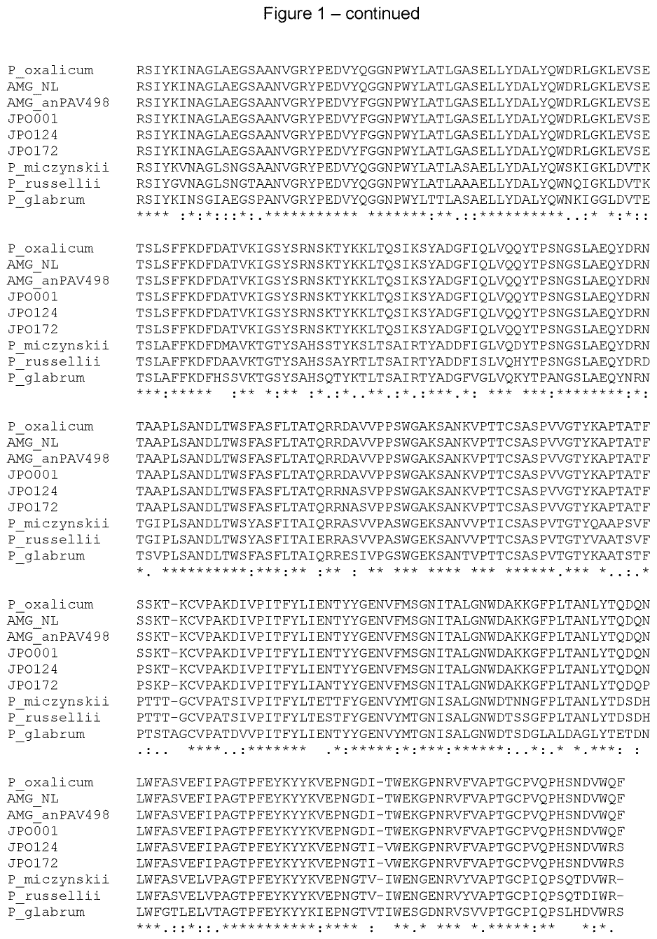

FIG. 1 shows a multiple alignment of the amino acid sequences of the mature proteins of:
Wildtype AMG from Penicillium oxalicum (PoAMG) of SEQ ID NO:1
PoAMG variant denoted ‘AMG NL’ of SEQ ID NO:2
PoAMG variant denoted ‘AMG anPAV498’ of SEQ ID NO:3
PoAMG variant denoted ‘AMG JPO001’ of SEQ ID NO:4
PoAMG variant denoted ‘AMG JPO124’ of SEQ ID NO:5
PoAMG variant denoted ‘AMG JPO172’ of SEQ ID NO:6
Wildtype AMG from Penicillium miczynskii (PoAMG) of SEQ ID NO:7
Wildtype AMG from Penicillium russellii (PoAMG) of SEQ ID NO:8
Wildtype AMG from Penicillium glabrum (PoAMG) of SEQ ID NO:9

### DETAILED DESCRIPTION OF THE INVENTION

### Definitions

In accordance with this detailed description, the following definitions apply. Note that the singular forms “a,” “an,” and “the” include plural references unless the context clearly dictates otherwise.

Throughout this document, values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1 percent to about 5 percent” or “about 0.1 percent to 5 percent” should be interpreted to include not just about 0.1 percent to about 5 percent, but also the individual values (e.g., 1 percent, 2 percent, 3 percent, and 4 percent) and the sub-ranges (e.g., 0.1 percent to 0.5 percent, 1.1 percent to 2.2 percent, 3.3 percent to 4.4 percent) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

Unless defined otherwise or clearly indicated by context, all technical and scientific terms used herein have the same meaning as commonly understood by one of ordinary skill in the art to which this invention belongs.
Glucoamylase: The term glucoamylase (1,4-alpha-D-glucan glucohydrolase, EC 3.2.1.3) is defined as an enzyme, which catalyzes the release of D-glucose from the non-reducing ends of starch or related oligo-and polysaccharide molecules. Glucoamylases are also called amyloglucosidases, and Glucan 1,4-alpha-glucosidase (EC 3.2.1.3), more commonly they are referred to as AMGs. The Glucoamylase Unit (AGU) is defined as the amount of enzyme, which hydrolyses 1 micromole maltose per minute under the standard conditions 37° C., pH 4.3, substrate: maltose 23.2 mM, buffer: acetate 0.1 M, reaction time 5 minutes.
Parent or parent glucoamylase: The term “parent” glucoamylase as used herein means a glucoamylase to which modifications are made to produce the variant glucoamylase of the present invention. This term also refers to the polypeptide with which a variant of the invention is compared. The parent may be a naturally occurring (wild type) polypeptide, or it may even be a variant thereof, prepared by any suitable means. For instance, the parent protein may be a variant of a naturally occurring polypeptide which has been modified or altered in the amino acid sequence. Thus, the parent glucoamylase may have one or more (or one or several) amino acid substitutions, deletions and/or insertions. Thus, the parent glucoamylase may be a variant of a parent glucoamylase. A parent may also be an allelic variant which is a polypeptide encoded by any of two or more alternative forms of a gene occupying the same chromosomal locus.
Mature polypeptide: The term “mature polypeptide” is defined herein as a polypeptide having biological activity that is in its final form following translation and any post-translational modifications, such as N-terminal processing, C-terminal truncation, glycosylation, phosphorylation, etc. The mature polypeptide sequence lacks a signal sequence, which may be determined using techniques known in the art (See, e.g., Zhang and Henzel, 2004, Protein Science 13:2819-2824). The term “mature polypeptide coding sequence” means a polynucleotide that encodes a mature polypeptide.
Sequence identity: The relatedness between two amino acid sequences or between two nucleotide sequences is described by the parameter “sequence identity”.

For purposes of the present invention, the sequence identity between two amino acid sequences is determined using the Needleman-Wunsch algorithm (Needleman and Wunsch, 1970, J. Mol. Biol. 48:443-453) as implemented in the Needle program of the EMBOSS package (EMBOSS: The European Molecular Biology Open Software Suite, Rice et al., 2000, Trends Genet. 16:276-277), preferably version 5.0.0 or later. The parameters used are gap open penalty of 10, gap extension penalty of 0.5, and the EBLOSUM62 (EMBOSS version of BLOSUM62) substitution matrix. The output of Needle labeled “longest identity” (obtained using the—no brief option) is used as the percent identity and is calculated as follows:

(Identical Residues×100)/(Length of Alignment−Total Number of Gaps in Alignment)
Variant: The term “variant” means a polypeptide having glucoamylase activity comprising an alteration, i.e., a substitution, insertion, and/or deletion, at one or more (e.g., several) positions. A substitution means replacement of the amino acid occupying a position with a different amino acid; a deletion means removal of the amino acid occupying a position; and an insertion means adding an amino acid adjacent to and immediately following the amino acid occupying a position. The variants of the present invention have at least 20%, e.g., at least 40%, at least 50%, at least 60%, at least 70%, at least 80%, at least 90%, at least 95%, or at least 100% of the glucoamylase activity of polypeptide of SEQ ID NO 1, SEQ ID NO: 6, SEQ ID NO: 7 or SEQ ID NO: 8.
Sensory Evaluation: To perform sensory evaluation, a panel (e.g., at least 3 well-trained persons) is used to assess the qualities of the steamed products.
Improved softness of the steamed product: The term “improved softness of the steamed product” is the opposite of “firmness/hardness” and is defined herein as the property of a steamed product that is more easily compressed compared to a control and is evaluated either empirically by well-trained persons with hand or mouth or measured by, e.g., a texture analyzer (e.g. TAXT2 or TA-XT Plus from Stable Micro Systems Ltd, surrey, UK) as known in the art.
Improved elasticity of the steamed product: The term “improved elasticity of the steamed product” is defined herein as the property of a steamed product that is more easily to recover to original status after pressed compared to a control and is evaluated either empirically by well-trained persons with hand or mouth or measured by, e.g., a texture analyzer (e.g. TAXT2 or TA-XT Plus from Stable Micro Systems Ltd, surrey, UK) as known in the art.
Improved appearance whiteness of the steamed product: The term “improved appearance whiteness of the steamed product” is defined herein as the property of a steamed product with whiter appearance in the surface compared to a control and is usually evaluated visually by well-trained persons.
Improved crumb structure of the steamed product: The term “improved crumb structure of the steamed product” is defined herein as the property of a steamed product with finer cells and/or thinner cell walls in the crumb and/or more uniform/homogenous distribution of cells in the crumb compared to a control and is usually evaluated visually by well-trained persons.
Improved moisture of the steamed product: The term “improved moisture of the steamed product” is defined herein as the property of a steamed product with cooling touch feel or cooling mouth feel compared to a control and is usually evaluated empirically by well-trained persons. Improved cohesiveness of steamed product: The term “improved cohesiveness of the steamed product” is defined herein as the property of a steamed product with less crumb falling compared to a control and is usually evaluated empirically by well-trained persons.
Improved chewiness of the steamed product: The term “improved chewiness of the steamed product” is defined herein as the property of a steamed product with more effort to chew before swallow compared to a control and is usually evaluated empirically by the skilled baker.
Improved sweetness of the steamed product: The term “improved sweetness of the steamed product” is defined herein as the property of a steamed product with sweeter taste compared to a control and is usually evaluated by well-trained persons.
Thermostability improvement: The thermostability improvement (Td) in C is a measure of how much the variants have improved in thermostability over their parent glucoamylase under the same conditions, determined as exemplified herein.

### Nomenclature

For purposes of the present invention, the nomenclature [Y/F] means that the amino acid at this position may be a tyrosine (Try, Y) or a phenylalanine (Phe, F). Likewise, the nomenclature [V/G/A/I] means that the amino acid at this position may be a valine (Val, V), glycine (Gly, G), alanine (Ala, A) or isoleucine (Ile, I), and so forth for other combinations as described herein. The amino acid X is defined such that it may be any of the 20 natural amino acids, unless otherwise stated.

### Conventions for Designation of Variants

For purposes of the present invention, the polypeptide disclosed in SEQ ID NO: 1 is used to determine the corresponding amino acid residue in another glucoamylase. Thus, all mentioned positions and specific substitutions refer to the numbering used in SEQ ID NO: 1. However, the skilled person would recognize that the sequence of any other sequence herein disclosed may also be used to determine the corresponding amino acid residue in another glucoamylase polypeptide. The amino acid sequence of another glucoamylase is aligned with the polypeptide disclosed in SEQ ID NO: 1, and based on the alignment, the amino acid position number corresponding the any amino acid residue in the polypeptide disclosed in SEQ ID No: 1 is determined using the Needleman-Wunsch algorithm (Needleman and Wunsch, 1970, J. Mol. Biol. 48:443-453) as implemented in the Needle program of the EMBOSS package (EMBOSS: The

European Molecular Biology Open Software Suite, Rice et al., 2000, Trends Genet. 16:276-277), preferably version 5.0.0 or later. The parameters used are gap open penalty of 10, gap extension penalty of 0.5, and the EBLOSUM62 (EMBOSS version of BLOSUM62) substitution matrix.

For proteins of known structure, several tools and resources are available for retrieving and generating structural alignments. For example, the SCOP superfamilies of proteins have been structurally aligned, and those alignments are accessible and downloadable. Two or more protein structures can be aligned using a variety of algorithms such as the distance alignment matrix (Holm and Sander, 1998, Proteins 33:88-96) or combinatorial extension (Shindyalov and Bourne, 1998, Protein Engineering 11:739-747), and implementation of these algorithms can additionally be utilized to query structure databases with a structure of interest in order to discover possible structural homologs (e.g., Holm and Park, 2000, Bioinformatics 16:566-567).

In describing the variants of the present invention, the nomenclature described below is adapted for ease of reference. The accepted IUPAC single letter or three letter amino acid abbreviation is employed.
Substitutions. For an amino acid substitution, the following nomenclature is used: Original amino acid, position, substituted amino acid. Accordingly, the substitution of threonine at position 226 with alanine is designated as “Thr226Ala” or “T226A”. Multiple mutations are separated by addition marks (“+”), e.g., “Gly205Arg+Ser411Phe” or “G205R+S411F”, representing substitutions at positions 205 and 411 of glycine (G) with arginine (R) and serine (S) with phenylalanine (F), respectively.
Deletions. For an amino acid deletion, the following nomenclature is used: Original amino acid, position, *. Accordingly, the deletion of glycine at position 195 is designated as “Gly195*” or “G195*”. Multiple deletions are separated by addition marks (“+”), e.g., “Gly195*+Ser411*” or “G195*+S411*”.
Insertions. For an amino acid insertion, the following nomenclature is used: Original amino acid, position, original amino acid, inserted amino acid. Accordingly the insertion of lysine after glycine at position 195 is designated “Gly195GlyLys” or “G195GK”. An insertion of multiple amino acids is designated [Original amino acid, position, original amino acid, inserted amino acid #1, inserted amino acid #2; etc.]. For example, the insertion of lysine and alanine after glycine at position 195 is indicated as “Gly195GlyLysAla” or “G195GKA”.

In such cases the inserted amino acid residue(s) are numbered by the addition of lower case letters to the position number of the amino acid residue preceding the inserted amino acid residue(s). In the above example, the sequence would thus be:

| Parent: | Variant: |
|---|---|
| 195 | 195 195a 195b |
| G | G - K - A |

Multiple alterations. Variants comprising multiple alterations are separated by addition marks (“+”), e.g., “Arg170Tyr+Gly195Glu” or “R170Y+G195E” representing a substitution of arginine and glycine at positions 170 and 195 with tyrosine and glutamic acid, respectively.
Different alterations. Where different alterations can be introduced at a position, the different alterations are separated by a comma, e.g., “Arg170Tyr, Glu” represents a substitution of arginine at position 170 with tyrosine or glutamic acid. Thus, “Tyr167Gly, Ala+Arg170Gly, Ala” designates the following variants:
   “Tyr167Gly+Arg170Gly”, “Tyr167Gly+Arg170Ala”, “Tyr167Ala+Arg170Gly”, and “Tyr167Ala+Arg170Ala”.

### DETAILED DESCRIPTION OF THE INVENTION

The first aspect relates to a method of producing a steamed flour-based product, comprising:
a) providing a dough or a paste comprising flour and a glucoamylase at least 70% identical to SEQ ID NO: 1, SEQ ID NO:6, SEQ ID NO:7 or SEQ ID NO:8; and
b) steaming the dough or the paste to make the steamed flour-based product.

In a preferred embodiment of the present invention, the said glucoamylase is a mature thermostable variant of a parent glucoamylase.

The second aspect of the invention relates to dough premix or paste premix comprising a dough or a paste, and a mature thermostable variant of a parent glucoamylase as defined in the present invention.

In a preferred embodiment of the present invention, compared with the same conditions as other conditions but without adding the enzyme to the dough/the paste, after 0.1, 0,25, 0.5, 1, 2, 3, 4, 5, 6 or 7 days of storage at room temperature or low temperature of 4° C., or stored up to 1, 2, 3, 4, 5, 6 months of −20° C., the steamed flour-based products of the invention have reduced hardness and/or improved elasticity, and thus have an improved storage stability.

In a preferred embodiment of the present invention, compared with the same conditions as other conditions but without adding the enzyme to the dough/the paste, after 0.1, 0,25, 0.5, 1, 2, 3, 4, 5, 6 or 7 days of storage at room temperature or low temperature of 4° C., or stored up to 1, 2, 3, 4, 5, 6 months of −20° C., the steamed flour-based product of the present invention has an improved sensory evaluation.

In a preferred embodiment of the present invention, compared with the same conditions as other conditions but without adding the enzyme to the dough/the paste, after 0.1, 0,25, 0.5, 1, 2, 3, 4, 5, 6 or 7 days of storage at room temperature or low temperature of 4° C., or stored up to 1, 2, 3, 4, 5, 6 months of −20° C., re-steaming the steamed flour-based products of the present invention, the re-steamed products have an improved sensory evaluation.

In a preferred embodiment of the present invention, the sensory evaluation is a comprehensive evaluation preferably, the average value, of softness, elasticity, appearance whiteness, crumb structure, moisture, cohesiveness, chewiness and/or sweetness the steamed flour-based products.

In another preferred embodiment of the present invention, the steamed flour-based product has at least the same sweetness or sweet taste as a control product made with double the amount of the mature glucoamylase the amino acid sequence of which is shown in SEQ ID NO: 10.

The other aspect relates to a method of producing a boiled flour-based product, comprising:
a) providing a dough comprising flour and a glucoamylase at least 70% identical to SEQ ID NO: 1, SEQ ID NO:6, SEQ ID NO:7 or SEQ ID NO:8; and
b) boiling the dough to make the boiled flour-based product.

In a preferred embodiment of the present invention, the said glucoamylase is a mature thermostable variant of a parent glucoamylase.

### Steamed Flour-Based Product:

As used herein, “steamed flour-based products” means any flour-based products prepared by steaming a dough or paste Examples of steamed flour-based products include steamed bread, such as Northern China steamed bread and southern China steamed bread, steamed steamed stuffed bun (bao zi), steamed twisted roll (hua juan), steamed roll (juan zi), steamed dumpling, braised noodles (men mian), spring festival cake (Nian Gao), steamed sponge cake (fa gao), or steamed sponge rice cake (mi fa gao). The steamed flour-based products may contain one or more additional ingredients, such, as meat (e.g., pork, beef, chicken or fish), vegetables (e.g., mushrooms, broccoli, and other green vegetables), fruits (e.g., dates and jujube), candies, cheese, and milk (or other dairy products), and combination thereof.

The present invention relates to a dough or paste comprising a thermostable glucoamylase of the present invention. As used herein “dough” means any dough used to prepare a steamed flour-based product such as a steamed bread. The dough used to prepare a steamed flour-based product may be made from any suitable flour source, e.g., flour sourced from grains, such as, wheat flour, corn flour, rye flour, barley flour, oat flour, rice flour, or sorghum flour, potato flour and combinations thereof (e.g., wheat flour combined with one of the other flour sources; rice flour combined with one of the other flour sources).

The dough of the present invention is usually a fermented dough or a dough to be fermented. The dough can be fermented in various ways, such as by adding dough ingredients such as chemical leavening agent (e.g. sodium bicarbonate) or by adding leavening agent (fermented dough), but it is preferable to ferment the dough by adding a suitable yeast culture, for example, a culture of Saccharomyces cerevisiae.

The present invention relates to a flour premix comprising a thermostable glucoamylase of the present invention, such flour premix may comprise any suitable flour source, e.g., flour sourced from grains, such as, wheat flour, corn flour, rye flour, barley flour, oat flour, rice flour, or sorghum flour, potato flour and combinations thereof (e.g., wheat flour combined with one of the other flour sources; rice flour combined with one of the other flour sources).

Methods of preparing steamed bread are well known in the art and include, for example, the “straight dough process” and the “sponge and dough process,” non-limiting examples of which are provided under the “Materials and Methods” section below. The process of preparing steamed bread generally involves the sequential steps of dough making (with an optional proofing step), sheeting, shaping, proofing, and then steaming the dough, which steps are well known in the art. If the optional proofing step is used, preferably more flour is added, and alkali may be added to neutralize acid produced or to be produced during the second proofing step.

Methods of preparing steamed sponge cake are well known in the art and include, for example, a method of preparing steamed sponge cake comprising preparing paste from materials containing flour, fermenting, and steaming to make the steamed sponge cake. Preferably, the paste is a rice paste. Preferably, the flour can be from grains, such as, wheat flour, corn flour, rye flour, barley flour, oat flour, rice flour, or sorghum flour, potato flour and combinations thereof.

Preferably, the finished cake has a delicate network structure, soft taste, pleasant wine aroma and lactic acid flavor produced by fermentation. Preferably, the paste contains a thermostable glucoamylase of the present invention. Preferably, the steamed sponge cake is made from rice, for example, the method comprising: soak the rice in water, and the soaked rice is ground and sieved to obtain rice paste, or a rice paste is made by adding water to steamed sponge cake premix powder, and the rice paste is fermented at 30-40° C. for around 1-25 hours, then sucrose and baking powder may be added, the mixture is evenly mixed, and steam for about 15 minutes to make the steamed sponge cake. The paste is generally fermented by the addition of a suitable yeast culture, for example, a culture of Saccharomyces cerevisiae (baker's yeast) or a chemical leavening agent, as are well-known in the art.

In addition to preparing fresh steamed bread dough or steamed bread products, the present invention is directed to method for preparing a frozen steamed bread dough. A frozen steamed bread dough may be advantageous for storage and/or distribution. An example of a method for preparing a frozen steamed bread dough includes the steps of making a dough (with an optional proofing), sheeting, shaping, proofing, and freezing the dough. The present invention is also directed to a frozen steamed bread dough comprising the thermostable glucoamylase of the present invention.

The present invention is particularly useful for preparing steamed bread dough and steamed bread products in industrialized processes, that is, in which the dough used to prepare steamed bread and/or steamed bread products are prepared mechanically using automated or semi-automated equipment. The present invention provides significant advantages in that steamed bread can now be prepared using automated or semi-automated processes in which the steamed bread is stored for distribution and consumer use more than 24 hours after preparation yet substantially maintains the qualities of steamed bread prepared freshly on the same day.

The process of preparing steamed bread generally involves the sequential steps of dough making (and an optional proofing step), sheeting, shaping, proofing, steaming and packaging. If the optional proofing step is used, preferably more flour is added, and alkali may be added to neutralize acid produced or to be produced during the second proofing step. In an industrial steamed bread production process according to the present invention, one or more of these steps, such as), sheeting, shaping, proofing, steaming and/or packaging, is/are performed using automated or semi-automated equipment.

In one embodiment, the glucoamylase according to the invention may be added to flour or dough or paste in an amount 0.01-1,000 mg enzyme protein (mg EP) per kg flour, preferably in an amount of 0.01-500 mg enzyme protein (mg EP) per kg flour, even more preferably in an amount of 0.1-100 mg enzyme protein (mg EP) per kg flour.

### Boiled Flour-Based Product:

As used herein, “Boiled flour-based products” means any flour-based products prepared by boiling dough. Examples of boiled flour-based products include traditional Chinese staple foods such as noodles, dumplings and wonton, rice dumplings and rice noodles, as well as dried products such as dried noodles and semi dry noodles, hot dry noodles, dry rice noodles and semi dry rice noodles as well as wet rice noodles. Flour may be made from any suitable flour source, e.g., flour sourced from grains, such as, rice flour, wheat flour, buckwheat flour, purple rice flour, corn flour, rye flour, barley flour, oat flour, or sorghum flour, potato flour and combinations thereof (e.g., wheat flour combined with one of the other flour sources; rice flour combined with one of the other flour sources). It can be a product with or without stuffing. The stuffing can be meat, vegetables, beans, or their combination. The dough of this kind of products is usually not fermented by yeast. The obtained dough can be processed into products of different shapes through different processes, with or without stuffing.

Boiled flour-based products are usually boiled at an appropriate time according to the thickness of the product or whether there is stuffing and frozen or dry state before eating. Different products are boiled in boiling water for 3-20 minutes to achieve the purpose of fully gelatinization.

In one embodiment, the glucoamylase according to the invention may be added to flour or dough in an amount 0.01-1,000 mg enzyme protein (mg EP) per kg flour, preferably in an amount of 0.01-500 mg enzyme protein (mg EP) per kg flour, even more preferably in an amount of 0.1-100 mg enzyme protein (mg EP) per kg flour.

### Glucoamylases

According to the present invention, different types of glucoamylases may be used as parent for the generation of a thermostable glucoamylase variant, e.g, the glucoamylase may be a polypeptide that is encoded by a DNA sequence that is found in a fungal strain of Aspergillus, Rhizopusor, Talaromyces or Penicillium; preferably the DNA sequence that is found in a fungal strain of Penicillium, even more preferably the DNA sequence that is found in a fungal strain of Penicillium oxysporum, Penicillium oxalicum, Penicillium miczynskii, Penicillium russellii or Penicillium glabrum. Examples of other suitable fungi include Aspergillus niger, Aspergillus awamori, Aspergillus oryzae, Rhizopus delemar, Rhizopus niveus, Rhizopus oryzae and Talaromyces emersonii.

The parent glucoamylase may be obtained from microorganisms of any genus. For purposes of the present invention, the term “obtained from” as used herein in the present invention in connection with a given source shall mean that the parent encoded by a polynucleotide is produced by the source or by a strain in which the polynucleotide from the source has been inserted. In one aspect, the parent is secreted extracellularly.

In one aspect of the present invention, the glucoamylase, or the parent glucoamylase, may be obtained from Penicillium such as, e.g., a Penicillium oxalicum, Penicillum glabrum, Penicillium brasilianum, Penicillium russellii, Penicillium miczynskii.

In one aspect, the parent fungal glucoamylase, may be a Penicillium glucoamylase such as, e.g., a Penicillium oxalicum glucoamylase, Penicillum glabrum glucoamylase, Penicillium brasilianum glucoamylase, Penicillium russellii glucoamylase, Penicillium miczynskii glucoamylase.

In another aspect, the parent glucoamylase is obtained from Penicillium oxalicum, e.g., shown as the glucoamylase of SEQ ID NO: 1. In another aspect, the parent glucoamylase is obtained from Penicillium oxalicum, e.g., shown as the glucoamylase of SEQ ID NO: 6. In another aspect, the parent glucoamylase is obtained from Penicillium oxalicum, e.g., shown as the glucoamylase of SEQ ID NO: 7. In another aspect, the parent glucoamylase is obtained from Penicillium oxalicum, e.g., shown as the glucoamylase of SEQ ID NO: 8.

In one embodiment, the thermostable glucoamylase of the invention is at least 70% identical to SEQ ID NO:1, SEQ ID NO:6, SEQ ID NO:7 or SEQ ID NO:8, e.g. at least 71%, e.g. at least 72%, e.g. at least 73%, e.g. at least 74%, e.g. at least 75%, e.g. at least 76%, e.g. at least 77%, e.g. at least 78%, e.g. at least 79%, e.g., at least 80%, e.g. at least 81%, e.g. at least 82%, e.g. at least 83%, e.g. at least 84%, e.g., at least 85%, e.g. at least 86%, e.g. at least 87%, e.g. at least 88%, e.g. at least 89%, e.g., at least 90%, e.g., at least 91%, e.g., at least 92%, e.g., at least 93%, e.g., at least 94%, e.g., at least 95%, e.g. at least 96%, e.g., at least 97%, e.g., at least 98%, e.g., at least 99% identical to SEQ ID NO: 1, SEQ ID NO:6, SEQ ID NO:7 or SEQ ID NO:8. In one embodiment, the glucoamylase is a glucoamylase variant or a mature thermostable variant of a parent glucoamylase.

In one embodiment, the mature variant comprises at least one amino acid modification in one or more (several) or all of the positions corresponding to positions 1, 2, 4, 6, 7, 11, 31, 34, 65, 79, 103, 132, 327, 445, 447, 481, 566, 568, 594 and 595 in SEQ ID NO:1.

In one embodiment, wherein the at least one amino acid modification comprises a substitution in one or more or all of the positions corresponding to positions 1, 2, 4, 11, 65, 79 and 327 in SEQ ID NO:1, preferably the at least one amino acid modification comprises a substitution in one or more or all of the positions corresponding to R1A, P2N, P4S, P11F, T65A, K79V and Q327F in SEQ ID NO:1.

In one embodiment, wherein the at least one amino acid modification comprises a substitution in one or more or all of the positions corresponding to positions 1, 6, 7, 31, 34, 79, 103, 132, 445, 447, 481, 566, 568, 594 and 595 in SEQ ID NO:1, preferably the at least one amino acid modification comprises a substitution in one or more or all of the positions corresponding to R1A, G6S, G7T, R31F, K34Y, K79V, S103N, A132P, D445N, V447S, S481P, D566T, T568V, Q594R and F595S in SEQ ID NO: 1.

In one embodiment, wherein the at least one amino acid modification comprises a substitution in one or more or all of the positions corresponding to positions 1, 6, 7, 31, 34, 50, 79, 103, 132, 445, 447, 481, 484, 501, 539, 566, 568, 594 and 595 in SEQ ID NO:1, preferably the at least one amino acid modification comprises a substitution in one or more or all of the positions corresponding to R1A, G6S, G7T, R31F, K34Y, E50R, K79V, S103N, A132P, D445N, V447S, S481P, T484P, E501A, N539P, D566T, T568V, Q594R and F595S in SEQ ID NO:1.

In one embodiment, wherein the mature thermostable variant has a thermostability improvement (Td) over its parent of at least 3° C., preferably at least 4° C., 5° C., 6° C., 7° C. or 8° C.

In one embodiment, wherein the mature thermostable variant has a relative activity at 91OC of at least 150, preferably at least 200, more preferably at least 250, most preferably at least 300 compared to its parent.

In one embodiment, the thermostable glucoamylase is a glucoamylase variant. In one embodiment, the glucoamylase variant of the present invention has a sequence identity to the polypeptide of SEQ ID NO: 1 or SEQ ID NO: 6, SEQ ID NO: 7 or SEQ ID NO: 8 of at least 60% e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, or 100%, which have glucoamylase activity.

In one embodiment, the amino acid sequence of the glucoamylase variant or the glucoamylase variant of the present invention differs by no more than ten amino acids, e.g., by nine amino acids, by eight amino acids, by seven amino acids, by six amino acids, by five amino acids, by four amino acids, by three amino acids, by two amino acids, and by one amino acid from SEQ ID NO: 1, SEQ ID NO: 6, SEQ ID NO: 7 or SEQ ID NO: 8 or the mature polypeptide of SEQ ID NO: 1, SEQ ID NO: 6, SEQ ID NO: 7 or SEQ ID NO: 8. In one embodiment. In one embodiment, the mature polypeptide of SEQ ID NO: 1, SEQ ID NO: 6, SEQ ID NO: 7 or SEQ ID NO: 8 is shown as SEQ ID NO: 1, SEQ ID NO: 6, SEQ ID NO: 7 or SEQ ID NO: 8.

In one embodiment, the amino acid sequence of the glucoamylase, the glucoamylase variant or the glucoamylase variant of the present invention comprises or consists of the amino acid sequence of SEQ ID NO: 1, SEQ ID NO: 6, SEQ ID NO: 7 or SEQ ID: 8.

The amino acid changes of variant may be of a minor nature, that is conservative amino acid substitutions or insertions that do not significantly affect the folding and/or activity of the protein; small deletions, typically of 1-30 amino acids; small amino- or carboxyl-terminal extensions, such as an amino-terminal methionine residue; a small linker peptide of up to 20-25 residues; or a small extension that facilitates purification by changing net charge or another function, such as a poly-histidine tract, an antigenic epitope or a binding domain.

Examples of conservative substitutions are within the groups of basic amino acids (arginine, lysine and histidine), acidic amino acids (glutamic acid and aspartic acid), polar amino acids (glutamine and asparagine), hydrophobic amino acids (leucine, isoleucine and valine), aromatic amino acids (phenylalanine, tryptophan and tyrosine), and small amino acids (glycine, alanine, serine, threonine and methionine). Amino acid substitutions that do not generally alter specific activity are known in the art and are described, for example, by H. Neurath and R. L. Hill, 1979, In, The Proteins, Academic Press, New York. Common substitutions are Ala/Ser, Val/Ile, Asp/Glu, Thr/Ser, Ala/Gly, Ala/Thr, Ser/Asn, Ala/Val, Ser/Gly, Tyr/Phe, Ala/Pro, Lys/Arg, Asp/Asn, Leu/Ile, Leu/Val, Ala/Glu, and Asp/Gly.

Alternatively, the amino acid changes are of such a nature that the physico-chemical properties of the polypeptides are altered. For example, amino acid changes may improve the thermal stability of the polypeptide, alter the substrate specificity, change the pH optimum, and the like.

Essential amino acids in a polypeptide can be identified according to procedures known in the art, such as site-directed mutagenesis or alanine-scanning mutagenesis (Cunningham and

Wells, 1989, Science 244:1081-1085). In the latter technique, single alanine mutations are introduced at every residue in the molecule, and the resultant mutant molecules are tested for glucoamylase activity to identify amino acid residues that are critical to the activity of the molecule. See also, Hilton et al., 1996, J. Biol. Chem. 271:4699-4708. The active site of the enzyme or other biological interaction can also be determined by physical analysis of structure, as determined by such techniques as nuclear magnetic resonance, crystallography, electron diffraction, or photoaffinity labeling, in conjunction with mutation of putative contact site amino acids. See, for example, de Vos et al., 1992, Science 255:306-312; Smith et al., 1992, J. Mol. Biol. 224:899-904; Wlodaver et al., 1992, FEBS Lett. 309:59-64. The identity of essential amino acids can also be inferred from an alignment with a related polypeptide.

Below is shown the %-identity between the AMG amino acid sequences aligned in FIG. 1, and also provided in the sequence list:

| | | | | | | | | | |
|---|---|---|---|---|---|---|---|---|---|
| P_oxalicum | 100.00 | 99.83 | 98.99 | 98.82 | 96.64 | 95.97 | 77.07 | 77.12 | 74.32 |
| AMG_NL | 99.83 | 100.00 | 99.16 | 98.99 | 96.81 | 96.13 | 77.07 | 77.12 | 74.32 |
| AMG_anPAV498 | 98.99 | 99.16 | 100.00 | 99.83 | 97.65 | 96.97 | 76.73 | 76.95 | 73.82 |
| AMG_JPO001 | 98.82 | 98.99 | 99.83 | 100.00 | 97.82 | 97.14 | 76.73 | 76.95 | 73.82 |
| AMG_JPO124 | 96.64 | 96.81 | 97.65 | 97.82 | 100.00 | 99.33 | 77.07 | 77.12 | 74.32 |
| AMG_JPO172 | 95.97 | 96.13 | 96.97 | 97.14 | 99.33 | 100.00 | 76.73 | 76.78 | 73.99 |
| P_miczynskii | 77.07 | 77.07 | 76.73 | 76.73 | 77.07 | 76.73 | 100.00 | 94.75 | 80.51 |
| P_russellii | 77.12 | 77.12 | 76.95 | 76.95 | 77.12 | 76.78 | 94.75 | 100.00 | 79.66 |
| P_glabrum | 74.32 | 74.32 | 73.82 | 73.82 | 74.32 | 73.99 | 80.51 | 79.66 | 100.00 |

Thermostable variants of the PoAMG have been generated (see table 2 below). In a preferred embodiment, the mature thermostable glucoamylase variant of the invention comprises one or more or all of the combinations of amino acid substitutions listed in table 2 below.

The thermostability improvements (Td) of the variants in table 2 are listed in Table 3, where the Td of the PoAMG variant denoted “anPAV498” (the parent) was set to zero. In a preferred embodiment, the the mature thermostable variant of the invention has a thermostability improvement (Td) over its parent of at least 3° C., preferably at least 4° C., 5° C., 6°° C., 7° C. or 8° C., preferably determined as exemplified herein.

In another preferred embodiment, the mature thermostable variant of the invention has a relative activity at 91° C. of at least 150, preferably at least 200, more preferably at least 250, most preferably at least 300 compared to its parent.

### Additional Enzymes

Optionally, one or more additional enzymes, such as alpha-amylase, maltogenic amylase, beta amylase, aminopeptidase, carboxypeptidase, catalase, cellulytic enzyme, chitinase, cutinase, cyclodextrin glycosyltransferase, deoxyribonuclease, esterase, glucan 1,4-alpha-maltotetrahydrolase, glucanase, galactanase, alpha-galactosidase, beta-galactosidase, glucose oxidase, alpha-glucosidase, beta-glucosidase, haloperoxidase, hemicellulytic enzyme, invertase, laccase, lipase, mannanase, mannosidase, oxidase, pectinolytic enzymes, peptidoglutaminase, peroxidase, phospholipase, phytase, polyphenoloxidase, proteolytic enzyme, ribonuclease, transglutaminase, and xylanase may be used together with the glucoamylase of the present invention.

The additional enzyme(s) may be of any origin, including mammalian, plant, and microbial (bacterial, yeast or fungal) origin.

### Enzyme Compositions

The mature thermostable glucoamylase variant of the invention as well as any additional enzyme(s) may be added to flour or dough in any suitable form, such as, e.g., in the form of a liquid, in particular a stabilized liquid, or it may be added to flour or dough as a substantially dry powder or granulate.

Granulates may be produced, e.g., as disclosed in U.S. Pa. Nos. 4, 106,991 and 4,661,452. Liquid enzyme preparations may, for instance, be stabilized by adding a sugar or sugar alcohol or lactic acid according to established procedures. Other enzyme stabilizers are well-known in the art.

The enzyme(s) may be added to the dough ingredients in any suitable manner, such as individual components (separate or sequential addition of the enzymes) or addition of the enzymes together in one step or one composition.

The invention described and claimed herein is not to be limited in scope by the specific embodiments herein disclosed, since these embodiments are intended as illustrations of several aspects of the invention. Any equivalent embodiments are intended to be within the scope of this invention as well as combinations of one or more of the embodiments.

Various references are cited herein, the disclosures of which are incorporated by reference in their entireties. The present invention is further described by the following example which should not be construed as limiting the scope of the invention.

### EXAMPLES

### EXAMPLE 1: Construction of PoAMG Libraries

### PoAMG Libraries Were Constructed as Follows:

A forward or reverse primer having NNK or desired mutation(s) at target site(s) with 15 bp overlaps each other were designed. Inverse PCR, which means amplification of entire plasmid DNA sequences by inversely directed primers, were carried out with appropriate template plasmid DNA (e.g. plasmid DNA containing JPO-0001 gene) by the following conditions. The resultant PCR fragments were purified by QIAquick Gel extraction kit [QIAGEN], and then introduced into Escherichia coli ECOS Competent E.coli DH5a [NIPPON GENE CO., LTD.]. The plasmid DNAs were extracted from E. coli transformants by MagExtractor plasmid extraction kit [TOYOBO], and then introduced into A. niger competent cells.

### PCR Reaction Mix:

PrimeSTAR Max DNA polymerase [TaKaRa]
Total 25 μl
1,0 μl Template DNA (1 ng/μl)
9,5 μl H₂O
12,5 μl 2× PrimeSTAR Max pre-mix
1,0 μl Forward primer (5 M)
1,0 μl Reverse primer (5 μM)

### PCR Program:

98° C./2 min
25×(98° C./10 sec, 60° C./15 sec, 72° C./2 min)
10° C./hold

### EXAMPLE 2: Screening for Better Thermostability

B. subtilis libraries constructed as in EXAMPLE 1 were fermented in either 96-well or 24-well MTP containing COVE liquid medium (2.0 g/L sucrose, 2.0 g/L iso-maltose, 2.0 g/L maltose, 4.9 mg/L, 0.2 ml/L 5 N NaOH, 10 ml/L COVE salt, 10 ml/L 1 M acetamide), 32° C. for 3 days. Then, AMG activities in culture supernatants were measured at several temperatures by pNPG assay described as follows.

pNPG Thermostability Assay:

The culture supernatants containing desired enzymes was mixed with same volume of pH 5.0 200 mM NaOAc buffer. Twenty microliter of this mixture was dispensed into either 96-well plate or 8-strip PCR tube, and then heated by thermal cycler at various temperatures for 30 min. Those samples were mixed with 10 μl of substrate solution containing 0.1% (w/v) pNPG [wako] in pH 5.0 200 mM NaOAc buffer and incubated at 70° C. for 20 min for enzymatic reaction. After the reaction, 60 μl of 0.1M Borax buffer was added to stop the reaction. Eighty microliter of reaction supernatant was taken out and its OD₄₀₅ value was read by photometer to evaluate the enzyme activity.

TABLE 1a
Lists of the relative activity of PoAMG variants
when compared with their parent anPAV498 or
JPO-0001 (anPAV498 w. leader-/propeptide)

| | Relative activity of |
|---|---|
| Name | 80° C. / 75° C. (%) |
| anPAV498 | 17% |
| JPO-004 | 32% |
| JPO-005 | 15% |
| JPO-006 | 16% |
| JPO-007 | 3% |

| | Relative activity of |
|---|---|
| Name | 80° C. / 75° C. (%) |
| AnPav498 | 13% |
| JPO-009 | 16% |
| JPO-011 | 15% |
| JPO-012 | 15% |
| JPO-013 | 17% |
| JPO-020 | 20% |

| | Relative activity of |
|---|---|
| Name | 80° C. / 70° C. (%) |
| JPO-001 | 10% |
| JPO-004 | 29% |
| JPO-009 | 13% |
| JPO-014 | 21% |
| JPO-020 | 16% |
| JPO-021 | 30% |
| JPO-052 | 33% |

| | Relative activity of |
|---|---|
| Name | 79° C. / 70° C. (%) |
| JPO-001 | 23% |
| JPO-021 | 46% |
| JPO-022 | 39% |
| JPO-023 | 44% |
| JPO-025 | 51% |
| JPO-027 | 49% |
| JPO-029 | 37% |

| | Relative activity of |
|---|---|
| Name | 77° C. / 70° C. (%) |
| JPO-001 | 72% |
| JPO-029 | 82% |
| JPO-047 | 80% |
| JPO-048 | 90% |
| JPO-049 | 84% |
| JPO-050 | 86% |
| JPO-064 | 87% |

| | Relative activity of |
|---|---|
| name | 79° C. / 77° C. (%) |
| JPO-001 | 36% |
| JPO-029 | 51% |
| JPO-047 | 45% |
| JPO-048 | 81% |
| JPO-049 | 53% |
| JPO-050 | 58% |
| JPO-064 | 65% |

| | Relative activity of |
|---|---|
| Name | 79° C. / 77° C. (%) |
| JPO-001 | 41% |
| JPO-021 | 60% |
| JPO-022 | 48% |
| JPO-023 | 57% |
| JPO-025 | 56% |
| JPO-027 | 64% |
| JPO-029 | 66% |
| JPO-047 | 50% |
| JPO-048 | 72% |
| JPO-051 | 82% |
| JPO-058 | 73% |
| JPO-062 | 72% |
| JPO-063 | 85% |
| JPO-064 | 83% |

TABLE 1b
Lists of the relative activity of PoAMG variants
when compared with their parent JPO-022

| | Relative activity of |
|---|---|
| Name | 77° C. / 70° C. (%) |
| JPO-022 | 60% |
| JPO-027 | 67% |
| JPO-042 | 8% |
| JPO-044 | 86% |
| JPO-045 | 67% |
| JPO-046 | 48% |

| | Relative activity of |
|---|---|
| Name | 77° C. / 70° C. (%) |
| JPO-022 | 76% |
| JPO-023 | 75% |
| JPO-025 | 80% |
| JPO-027 | 84% |
| JPO-058 | 92% |
| JPO-059 | 88% |
| JPO-060 | 86% |
| JPO-061 | 83% |
| JPO-062 | 87% |

| | Relative activity of |
|---|---|
| Name | 79° C. / 77° C. (%) |
| JPO-022 | 49% |
| JPO-023 | 51% |
| JPO-025 | 52% |
| JPO-027 | 58% |
| JPO-058 | 69% |
| JPO-059 | 36% |
| JPO-060 | 41% |
| JPO-061 | 44% |
| JPO-062 | 57% |

TABLE 1c
List of the relative activity of PoAMG variants
when compared with their parent JPO-063

| | Relative activity of |
|---|---|
| Name | 79° C. / 77° C. (%) |
| JPO-063 | 91% |
| JPO-066 | 96% |
| JPO-071 | 89% |
| JPO-072 | 84% |
| JPO-074 | 103% |
| JPO-075 | 86% |
| JPO-076 | 92% |
| JPO-077 | 95% |
| JPO-078 | 88% |
| JPO-079 | 100% |

| | Relative activity of |
|---|---|
| Name | 84° C. / 80° C. (%) |
| JPO-063 | 16% |
| JPO-065 | 26% |
| JPO-067 | 21% |
| JPO-070 | 12% |
| JPO-071 | 13% |
| JPO-074 | 32% |
| JPO-081 | 17% |
| JPO-082 | 24% |
| JPO-083 | 46% |
| JPO-084 | 26% |
| JPO-044 | 37% |

| Name | Relative activity of 82° C./70° C. (%) |
|---|---|
| JPO-063 | 21% |
| JPO-093 | 43% |
| JPO-081 | 25% |
| JPO-088 | 39% |
| JPO-094 | 38% |
| JPO-096 | 38% |
| JPO-106 | 53% |

| Name | Relative activity of 83° C./80° C. (%) |
|---|---|
| JPO-063 | 46% |
| JPO-051 | 44% |
| JPO-096 | 64% |
| JPO-106 | 88% |
| JPO-110 | 81% |
| JPO-111 | 100% |
| JPO-112 | 86% |
| JPO-113 | 83% |
| JPO-114 | 47% |
| JPO-115 | 90% |

TABLE 1d
List of the relative activity of PoAMG variants
when compared with their parent JPO-096

| Name | Relative activity of 83° C./70° C. (%) |
|---|---|
| JPO-082 | 53% |
| JPO-088 | 70% |
| JPO-091 | 69% |
| JPO-092 | 65% |
| JPO-093 | 62% |
| JPO-094 | 74% |
| JPO-095 | 69% |
| JPO-096 | 67% |
| JPO-097 | 65% |
| JPO-098 | 65% |

| Name | Relative activity of 83° C./80° C. (%) |
|---|---|
| JPO-051 | 20% |
| JPO-096 | 43% |
| JPO-109 | 51% |
| JPO-126 | 33% |
| JPO-129 | 48% |
| JPO-130 | 18% |
| JPO-131 | 51% |
| JPO-132 | 34% |

TABLE 1e
List of the relative activity of PoAMG variants
when compared with their parents JPO-129

| Name | Relative activity of 84° C./80° C. (%) |
|---|---|
| JPO-129 | 62% |
| JPO-156 | 51% |
| JPO-160 | 34% |
| JPO-161 | 41% |
| JPO-162 | 49% |
| JPO-163 | 21% |
| JPO-164 | 57% |
| JPO-165 | 77% |

TABLE 1f
List of the relative activity of PoAMG variants
when compared with their parent JPO-166

| Name | Relative activity of 84° C./75° C. (%) |
|---|---|
| JPO-166 | 19% |
| JPO-167 | 66% |
| JPO-168 | 58% |
| JPO-169 | 53% |
| JPO-171 | 47% |
| JPO-172 | 98% |

TABLE 2
Amino acid substitutions in the variants of the PoAMG mature sequence

| Name | Amino acid substitutions |
|---|---|
| PoAMG | The wildtype mature AMG from Penicillium (SEQ ID NO: 1) |
| AMG NL | K79V |
| anPAV498 | P2N P4S P11F T65A K79V Q327F |
| JPO-001 | R1A P2N P4S P11F T65A K79V Q327F |
| JPO-018 | D75N R77D A78Q |
| JPO-019 | D75S R77G A78W V79D F80Y |
| JPO-023 | R1A K34Y S103N |
| JPO-024 | R1A K34Y D445N V447S |
| JPO-025 | R1A K34Y Y504T |
| JPO-026 | R1A S103N D445N V447S |
| JPO-027 | R1A S103N Y504T |
| JPO-028 | R1A D445N V447S Y504T |
| JPO-029 | R1A K34Y S103N D445N V447S |
| JPO-044 | R1A K34Y S103N D445N V447S E501V Y504T |
| JPO-047 | R1A K34Y S103N Y504T |
| JPO-048 | R1A K34Y S103N D445N V447S D566T |
| JPO-049 | R1A K34Y S103N Q594R F595S |
| JPO-050 | R1A K34Y S103N Y504T Q594R F595S |
| JPO-051 | R1A K34Y S103N D445N V447S Y504T Q594R F595S |
| JPO-052 | R1A S105L |
| JPO-053 | R1A S105E |
| JPO-055 | R1A A132R |
| JPO-058 | R1A K34Y S105L Y504T Q594R F595S |
| JPO-059 | R1A K34Y S103N S105L Y504T Q594R F595S |
| JPO-060 | R1A K34Y S103N S105L Y504T Q594R F595S |
| JPO-061 | R1A K34Y S103N S105L Y504T D566T Q594R F595S |
| JPO-062 | R1A K34Y S103N S105L D445N V447S Y504T D566T Q594R F595S |
| JPO-063 | R1A K34Y S103N S105L A132R D445N V447S Y504T D566T Q594R F595S |
| JPO-064 | R1A K34Y S103N S105L D445N V447S D566T Q594R F595S |
| JPO-065 | R1A K34Y S103N S105L A132R D445N V447S E501V Y504T D566T Q594R R1A |
| | F595S |
| JPO-066 | R1A K34Y S103N A132R D445N V447S Y504T D566T Q594R F595S |
| JPO-069 | R1A K34Y S103N S105L A132R D445N V447S Y504T D566T V592T |
| JPO-071 | R1A G6S G7T K34Y S103N S105L A132R D445N V447S Y504T D566T Q594R |
| | R1A F595S |
| JPO-074 | R1A K34Y S103N P107L A132R D445N V447S Y504T D566T Q594R F595S |
| JPO-083 | R1A G6S G7T K34Y S103N P107L A132R D445N V447S Y504T D566T Q594R |
| | R1A F595S |
| JPO-084 | R1A G6S G7T K34Y S103N P107L A132R D445N V447S Y504T D566T V592T |
| | R1A Q594R F595S |
| JPO-091 | R1A G6S R7T K34Y S103N P107L A132P D445N V447S Y504T D566T Q594R |
| | F595S |
| JPO-092 | R1A G6S G7T K34Y S103N P107L A132R D445N V447S Y504T D566T T568V |
| | Q594R F595S |
| JPO-093 | R1A G6S G7T K34Y S103N P107L A132P D445N V447S Y504T D566T T568V |
| | Q594R F595S |
| JPO-094 | R1A G6S G7T K34Y S103N P107L A132R D445N V447S S481P Y504T D566T |
| | Q594R F595S |
| JPO-095 | R1A G6S G7T K34Y S103N P107L A132R D445N V447S S481P Y504T D566T |
| | T568V Q594R F595S |
| JPO-096 | R1A G6S G7T K34Y S103N P107L A132P D445N V447S D566T T568V Q594R |
| | F595S |
| JPO-097 | R1A G6S G7T K34Y S103N P107L T110W A132P D445N V447S Y504T D566T |
| | T568V Q594R F595S |
| JPO-098 | R1A G6S G7T K34Y E50R S103N P107L A132P D445N V447S Y504T D566T |
| | T568V Q594R F595S |
| JPO-105 | R1A G6S G7T K34Y S103N P107L A132P D445N V447S E501V Y504T |
| JPO-106 | R1A G6S G7T R31F K34Y S103N P107L A132P D445N V447S Y504T D566T |
| | T568V Q594R F595S |
| JPO-108 | R1A G6S G7T R31F K34Y S103N P107L A132P D445N V447S S481P Y504T |
| | D566T T568V Q594R F595S |
| JPO-109 | R1A G6S G7T K34Y E50R S103N P107L A132P D445N V447S S481P Y504T |
| | D566T T568V Q594R F595S |
| JPO-111 | R1A G6S G7T R31F K34Y S103N P107L A132P D445N V447S S481P E501V |
| | Y504T D566T T568V Q594R F595S |
| JPO-112 | R1A G6S G7T R31F K34Y S103N P107L A132P D445N V447S S481P D566T |
| | T568V Q594R F595S |
| JPO-114 | R1A K34Y D75N R77D A78Q S103N R138L D445N V447S Y504T Q594R F595S |
| JPO-115 | R1A G6S G7T R31F K34Y D75N R77D A78Q S103N P107L A132P D445N |
| | V447S S481P Y504T D566T T568V Q594R F595S |
| JPO-124 | R1A G6S G7T R31F K34Y S103N A132P D445N V447S S481P D566T T568V |
| | Q594R F595S |
| JPO-125 | R1A G6S G7T K34Y E50R S103N A132P D445N V447S S481P D566T T568V |
| | Q594R F595S |
| JPO-126 | R1A R31F K34Y D75N R77D A78Q S103N R138L D445N V447S Y504T Q594R |
| | F595S |
| JPO-127 | R1A K34Y D75N R77D A78Q S103N R138L D445N V447S Q594R F595S |
| JPO-128 | R1A G6S G7T R31F K34Y S103N A132P D445N V447S |
| JPO-129 | R1A G6S G7T R31F K34Y E50R S103N A132P D445N V447S S481P D566T |
| | T568V Q594R F595S |
| JPO-130 | R1A K34Y E50R D75N R77D A78Q S103N R138L D445N V447S Q594R F595S |
| JPO-131 | R1A G6S G7T R31F K34Y E50R D75N R77D A78Q S103N A132P D445N V447S |
| | S481P D566T Q594R F595S |
| JPO-132 | R1A R31F K34Y E50R D75N R77D A78Q S103N R138L D445N V447S Q594R |
| | F595S |
| JPO-133 | R1A G6S G7T R31F K34Y E50R D75N R77D A78Q S103N A132P R138L D445N |
| | V447S S481P D566T Q594R F595S |
| JPO-138 | R1A R135S |
| JPO-143 | R1A G6S G7T R31F K34Y E50R S103N A132P D445N V447S S481P E501L |
| | D566T T568V Q594R F595S |
| JPO-154 | R1A G6S G7T R31F K34Y S103N A132P R138G D445N V447S S481P D566T |
| | T568V Q594R F595S |
| JPO-155 | R1A G6S G7T R31F K34Y S103N A132P R138L D445N V447S S481P D566T |
| | T568V Q594R F595S |
| JPO-156 | R1A G6S G7T R31F K34Y S103N A132P R138P D445N V447S S481P D566T |
| | T568V Q594R F595S |
| JPO-167 | R1A G6S G7T R31F K34Y E50R S103N A132P S379P D445N V447S S481P |
| | E501A D566T T568V Q594R F595S |
| JPO-168 | R1A G6S G7T R31F K34Y E50R S103N A132P D445N V447S S481P T484P |
| | E501A D566T T568V Q594R F595S |
| JPO-169 | R1A G6S G7T R31F K34Y E50R S103N A132P D445N V447S S481P E501A |
| | N539P D566T T568V Q594R F595S |
| JPO-171 | R1A G6S G7T R31F K34Y E50R S103N A132P S379P D445N V447S S481P |
| | T484P E501A D566T T568V Q594R F595S |
| JPO-172 | R1A G6S G7T R31F K34Y E50R S103N A132P D445N V447S S481P T484P |
| | E501A N539P D566T T568V Q594R F595S |

### EXAMPLE 3: Fermentation of the Aspergillus niger

Aspergillus niger strains were fermented on a rotary shaking table in 500 ml baffled flasks containing 100 ml MU1 with 4 ml 50% urea at 220 rpm, 30° C. The culture broth was centrifuged (10,000×g, 20 min) and the supernatant was carefully decanted from the precipitates.

### EXAMPLE 4: Purification of PoAMG (JPO-001) Variants

PoAMG variants were purified by cation exchange chromatography. The peak fractions of each were pooled individually and dialyzed against 20 mM sodium acetate buffer pH 5.0, and then the samples were concentrated using a centrifugal filter unit (Vivaspin Turbo 15, Sartorius). Enzyme concentrations were determined by A280 value.

### EXAMPLE 5: Thermostability Determination (TSA)

Purified enzyme was diluted with 50 mM sodium acetate buffer pH 5.0 to 0.5 mg/ml and mixed with equal volume of SYPRO Orange (Invitrogen) diluted with Milli-Q water. Eighteen ul of mixture solution were transfer to LightCycler 480 Multiwell Plate 384 (Roche Diagnostics) and the plate was sealed.

### Equipment Parameters of TSA:

Apparatus: LightCycler 480 Real-Time PCR System (Roche Applied Science)
Scan rate: 0.02° C./sec
Scan range: 37-96° C.
Integration time: 1.0 sec
Excitation wave length 465 nm
Emission wave length 580 nm

The obtained fluorescence signal was normalized into a range of 0 and 1. The Td was defined as the temperature at which the signal intensity was 0.5. The thermostability improvements are listed in Table 3 with Td of the PoAMG variant denoted anPAV498 as 0.

### EXAMPLE 6: PoAMG Activity Assay

Maltodextrin (DE11) assay by GOD-POD method

### Substrate Solution

30 g maltodextrin (pindex #2 from MATSUTANI chemical industry Co., Ltd.)
100 ml 120 mM sodium acetate buffer, pH 5.0
Glucose CII test kit (Wako Pure Chemical Industries, Ltd.)

Twenty ul of enzyme samples were mixed with 100 ul of substrate solution and incubated at set temperatures for 2 hours. The samples were cooled down on the aluminum block for 3 min then 10 ul of the reaction solution was mixed with 590 ul of 1 M Tris-HCl PH 8.0 to stop reaction. Ten ul of the solution was mixed with 200 ul of the working solution of the test kit then stand at room temperature for 15 min. The absorbance at A505 was read. The activities are listed in Table 3 as relative activity of the PoAMG variant denoted anPAV498.

TABLE 3

| | Td improvement [° C.] | Activity at 91° C. |
|---|---|---|
| Variant | (pH 5.0, anPAV498 as 0) | (anPAV498 as 100) |
| anPAV498 | — | 100 |
| JPO-001 | 1.0 | 94 |
| JPO-004 | 2.2 | — |
| JPO-009 | 0.7 | — |
| JPO-013 | 1.5 | — |
| JPO-014 | 2.3 | — |
| JPO-020 | 1.4 | 74 |
| JPO-021 | 2.5 | 113 |
| JPO-052 | 2.6 | 85 |
| JPO-053 | 0.2 | 71 |
| JPO-055 | 1.6 | 85 |
| JPO-023 | 3.6 | — |
| JPO-024 | 2.5 | — |
| JPO-025 | 3.4 | — |
| JPO-027 | 2.9 | — |
| JPO-029 | 3.7 | 191 |
| JPO-048 | 4.3 | 163 |
| JPO-051 | 5.7 | 222 |
| JPO-058 | 4.2 | 157 |
| JPO-062 | 4.2 | 159 |
| JPO-063 | 5.4 | 107 |
| JPO-064 | 4.9 | 178 |
| JPO-065 | 7.0 | 127 |
| JPO-066 | 6.5 | 178 |
| JPO-069 | 4.8 | 95 |
| JPO-071 | 6.1 | 128 |
| JPO-074 | 6.3 | 108 |
| JPO-081 | 5.5 | 213 |
| JPO-082 | 5.6 | 215 |
| JPO-089 | 6.0 | 171 |
| JPO-090 | 5.5 | 155 |
| JPO-018 | 0.6 | 84 |
| JPO-019 | 0.5 | 86 |
| JPO-044 | 6.3 | 225 |
| JPO-083 | 6.1 | 103 |
| JPO-084 | 4.4 | 66 |
| JPO-099 | 6.8 | 156 |
| JPO-091 | 6.6 | 130 |
| JPO-092 | 6.7 | 113 |
| JPO-093 | 6.8 | 132 |
| JPO-094 | 6.6 | 126 |
| JPO-095 | 6.9 | — |
| JPO-096 | 5.9 | — |
| JPO-097 | 5.2 | — |
| JPO-098 | 5.6 | — |
| JPO-112 | 8.2 | — |
| JPO-114 | 5.2 | 218 |
| JPO-115 | 8.0 | — |
| JPO-108 | 8.5 | — |
| JPO-109 | 7.2 | — |
| JPO-111 | 8.4 | — |
| JPO-124 | 8.0 | 385 |
| JPO-125 | 6.8 | 324 |
| JPO-126 | 6.6 | 268 |
| JPO-127 | 4.9 | 246 |
| JPO-129 | 8.2 | 399 |
| JPO-130 | 5.3 | 278 |
| JPO-131 | 7.9 | 367 |
| JPO-132 | 6.6 | 336 |
| JPO-138 | 6.4 | 125 |
| JPO-133 | 6.1 | 143 |
| JPO-143 | 8.8 | 280 |
| JPO-154 | 7.6 | 252 |
| JPO-155 | 8.3 | 282 |
| JPO-156 | 8.3 | 290 |
| JPO-145 | 8.2 | — |
| JPO-147 | 8.2 | — |
| JPO-150 | 8.2 | — |
| JPO-152 | 8.4 | — |
| JPO-153 | 9.0 | 399 |
| JPO-161 | 6.0 | 200 |
| JPO-165 | 8.9 | 403 |
| JPO-166 | 7.0 | 237 |
| JPO-167 | 9.1 | 387 |
| JPO-168 | 9.3 | 332 |
| JPO-169 | 9.6 | 269 |
| JPO-171 | 9.4 | 255 |
| JPO-172 | 9.9 | 432 |

### Example 7: Use of PoAMG in Steamed Bread

Northern China steamed bread was prepared by a Straight dough process with a recipe according to Table 4 and Table 5. All raw materials used herein are food grade, PoAMG variant JPO-172 (75 ppm used herein represents 24.45 mg EP/1000 g flour) and maltogenic alpha-amylase were used, maltogenic alpha-amylase herein is Novamyl Boost (commercial product of Novozymes). Briefly, flour, yeast and steamed bread improver were weighed and put into a dough jar (vertical mixer, DIOSNA brand), then enzyme and water were added. The mixture was stirred at a low speed for 6 minutes until the dough was formed and the dough surface was smooth, and the dough was sheeted till it reached the ideal degree. The sheeting times is dependent on hand feeling. After sheeting, dough was weighed out around 110 g and molded it into a steamed bread shape. The molded dough was put into proofing machine for around 40 min under 35° C. (room humidity around 80%). After proofing, the dough was put in a steamer (100° C.) and was steamed for 20 min. Then the steam was turned off, about 5 mins later, the steamed breads were taken out and cooled at room temperature for 2 hours, after that the prepared steamed bread was packaged with a sealed plastic package, for texture and sensory evaluation.

Re-steaming of steamed bread: The prepared steamed bread was stored at room temperature for 24 hrs or in refrigerator at 4° C. for 48 hrs, and then it was re-steamed in a steamer (100° C.) for around 15 mins. Then the steam was turned off, about 5 mins later, the re-steamed steamed bread was taken out for sensory evaluation.

TABLE 4
Recipe of Northern Steamed Bread

| | | weigh percentage % (based |
|---|---|---|
| Recipe | Commercial supplier | on flour) |
| wheat flour | Wudeili, China | 100 |
| Yeast (high activity dry yeast) | Angel Yeast | 0.8 |
| Steamed bread improver | Angel Yeast (haodadang | 0.5 |
| | brand) | |
| water | | 50-52 |
| JPO-172 | / | See Table 5 |
| Novamyl Boost | Novozymes A/S | |

TABLE 5

| Batch | JPO-172 | Novamyl Boost |
|---|---|---|
| A | 0 | 0 |
| B | 75 ppm | 0 |
| C | 0 | 100 ppm |

### Sample Characterization

Method for determining hardness: The steamed bread was divided by using a slicer (the thickness of each steamed bread slice was 1.2 cm), two sliced steamed bread slices were in one group (the thickness was 2.4 cm), and were determined by a TA.XT Plus texture analyzer. Gram is used as the unit, the higher the hardness value, represents that the quality of the prepared steamed bread is worse.

Method for determining elasticity: The steamed bread was divided by using a slicer (the thickness of each steamed bread slice was 1.2 cm). Two sliced steamed bread slices were in one group (the thickness was 2.4 cm), and were determined by a TA.XT Plus texture analyzer. % was used as the unit. The higher the elasticity value, represents that the quality of the prepared steamed bread is better.

### Sensory Evaluation:

To perform sensory evaluation, a panel (5 well-trained persons) was used to assess the qualities of the steamed bread/the re-steamed steamed bread. Parameters, such as, softness, elasticity, appearance whiteness, crumb structure, moisture, cohesiveness, chewiness and/or sweetness were scored. The steamed bread prepared in batch A was scored as 5.0 points and used as the baseline. The mean was taken for a comprehensive evaluation, wherein the higher the score of the mean, represented that the quality of the prepared steamed bread was better.

TABLE 6
Hardness and elasticity of steamed bread after
being stored for 24 hrs at room temperature

| Batch | Hardness/g | Elasticity/% |
|---|---|---|
| A | 2655 | 54.96 |
| B | 1512 | 63.21 |
| C | 1691 | 60.67 |

As can be seen from table 6, compared with the control (Batch A), when PoAMG (JPO-172) and Novamyl Boost were used during the preparation of the steamed bread, the hardness of the steamed breads were both significantly reduced, while the elasticity of the steamed bread also were both improved. However, in contrast,] PoAMG (JPO-172) showed a better effect than Novamyl Boost.

Each batch of the steamed breads prepared were subjected to sensory evaluation after being stored at room temperature for 24 hr hours, and the results are shown in Table 7. Compared with the control (Batch A), the addition of PoAMG (JPO-172) and Novamyl Boost in the dough improved the sensory evaluation of steamed bread, such as the touch softness, touch elasticity, crumb structure, moisture, cohesiveness, and chewiness were also improved. In addition, PoAMG (JPO-172) also improved the sweetness of steamed bread. In contrast, PoAMG (JPO-172) was significantly better than Novamyl Boost in improving the quality of steamed bread.

TABLE 7
Sensory evaluation of steamed bread (after 24 hrs at room temperature)

| | Touch | Touch | Appearance | Crumb | | | | | |
|---|---|---|---|---|---|---|---|---|---|
| Batch | softness | elasticity | whiteness | structure | Moisture | Cohesiveness | Chewiness | Sweetness | Average |
| A | 5.0 | 5.0 | 5.0 | 5.0 | 5.0 | 5.0 | 5.0 | 5.0 | 5.0 |
| B | 6.3 | 6.0 | 4.8 | 5.1 | 5.5 | 5.7 | 5.7 | 5.7 | 5.6 |
| C | 5.8 | 5.5 | 4.3 | 4.9 | 5.6 | 5.7 | 5.7 | 5.3 | 5.3 |

Each batch of steamed bread prepared were stored at room temperature for 24 hours and then were re-steamed, and then let them cool down to room temperature, after that such re-steamed steamed breads were subjected to sensory evaluation and the results was shown in Table 8.

TABLE 8
Sensory evaluation of steamed bread (re-steaming
after storing at room temperature for 24 hrs)

| | Touch | Touch | Apperance | Crumb | | | |
|---|---|---|---|---|---|---|---|
| | softness | elasticity | whiteness | structure | Chewiness | Sweetness | Average |
| A | 5.0 | 5.0 | 5.0 | 5.0 | 5.0 | 5.0 | 5.0 |
| B | 5.6 | 5.6 | 5.5 | 5.6 | 5.0 | 5.5 | 5.5 |
| C | 4.8 | 4.8 | 4.4 | 4.6 | 4.8 | 5.0 | 4.7 |

Each batch of steamed bread prepared were stored at 4°° C. for 48 hours and then were re-steamed, and then let them cool down to room temperature, after that such re-steamed steamed breads were subjected to sensory evaluation and the results was shown in Table 9.

TABLE 9
Sensory evaluation of steamed bread (re-steaming
after storage at 4° C. for 48 hours)

| | Touch | Touch | Appearance | Crumb | | | |
|---|---|---|---|---|---|---|---|
| | softness | elasticity | whiteness | structure | Chewiness | Sweetness | Average |
| A | 5.0 | 5.0 | 5.0 | 5.0 | 5.0 | 5.0 | 5.0 |
| B | 5.5 | 5.5 | 5.5 | 5.3 | 5.2 | 5.4 | 5.4 |
| C | 4.9 | 5.0 | 4.4 | 5.1 | 4.7 | 5.0 | 4.8 |

As can be seen from Table 8 and Table 9, compared with the control Batch A, the addition of PoAMG (JPO-172) in the dough obviously improved the sensory evaluation of the re-steamed steamed bread, such as the touch softness, touch elasticity, crumb structure, chewiness and sweetness were all improved. PoAMG (JPO-172) also improved the appearance whiteness of the re-steamed steamed bread. However, in contrast, even when Novamyl Boost was added, the sensory evaluation of re-steamed steamed bread was not good, it was not even as good as that of batch A.

### Example 8: Use of POAMG in Steamed Sponge Rice Cake

Steamed sponge rice cake was prepared with a recipe according to Table 10 and Table 11, briefly, rice paste was prepared by mixing steamed sponge rice cake premix powder and water in a container, batch A was control without enzyme, PoAMG (JPO-172) was added in the rice paste as Batch B. The mixture was sealed and fermented at 35° C. for 16 hours, then sucrose and baking powder were added after fermentation. After that, the fermented mixture was stirred and put in a mold, and then was steamed in a steamer (100° C.) for around 15 minutes. Then the steam was turned off, about 5 mins later, the steamed sponge rice cakes were taken out and cooled at room temperature for 3 hours for texture and sensory evaluation.

TABLE 10
Recipe of steamed sponge rice cake

| | weigh percentage % (based on |
|---|---|
| Recipe | Premix powder) |
| steamed sponge rice cake premix powder | 100 |
| (commercially available from Angel | |
| Yeast) | |
| Water | 100 |
| Sucrose | 17 |
| Baking powder | 2 |

TABLE 11

| Batch | Content |
|---|---|
| A | 0 |
| B | 65 mg EP/1000 g Premix powder |

Method for determining hardness: cut a flat surface of steamed sponge rice cake along the outer edge of the mold (the height of each sample was 2.5 cm), and the hardness was determined by a TA.XT Plus texture analyzer. Gram is used as the unit, the higher the hardness value, represents that the quality of the prepared product is worse.

Method for determining elasticity: cut a flat surface of steamed sponge rice cake along the outer edge of the mold (the height of each sample was 2.5 cm), and the elasticity were determined by a TA.XT Plus texture analyzer. The higher the elasticity value, represents that the quality of the prepared product is better.

### Sensory Evaluation:

To perform sensory evaluation, a panel (5 well-trained persons) was used to assess the qualities of the steamed sponge rice cakes. Parameters, such as, mouthfeel softness, mouthfeel moisture, mouthfeel elasticity, crumb structure, and sweetness were scored. The steamed sponge rice cake prepared in batch A was scored as 5.0 points and used as the baseline. The mean was taken for a comprehensive evaluation, wherein the higher the score of the mean, represented that the quality of the prepared steamed sponge cake was better.

The sensory evaluation of the prepared steamed sponge rice cakes is shown in Table 12. When PoAMG (JPO-172) was added in the paste, mouthfeel softness, mouthfeel moisture, mouthfeel elasticity and sweetness of the prepared steamed sponge cake were improved without crumb structure loss.

TABLE 12
Sensory evaluation of steamed sponge rice cake

| | mouthfeel | mouthfeel | Mouthfeel | crumb | |
|---|---|---|---|---|---|
| Batch | softness | moisture | elasticity | structure | Sweetness |
| A | 5 | 5 | 5 | 5 | 5 |
| B | 6.4 | 6.1 | 5.9 | 5 | 5.5 |

In addition, the results from the texture analysis can be found in Table 13, when PoAMG (JPO-172 was used in the preparation of the steamed sponge rice cake, the prepared steamed sponge rice cake had reduced hardness and improved elasticity.

TABLE 13
Hardness and elasticity

| Batch | Hardness/g | Elasticity/% |
|---|---|---|
| A | 3435.25 | 74.18 |
| B | 1810.09 | 77.08 |

1. A method of producing a steamed flour-based product, said method comprising:
a) providing a dough or a paste comprising flour and a glucoamylase at least 70% identical to SEQ ID NO: 1, SEQ ID NO: 6, SEQ ID NO: 7 or SEQ ID NO: 8; and
b) steaming the dough to produce a steamed flour-based product.

2. The method according to claim 1, wherein the glucoamylase is a glucoamylase variant or a mature thermostable variant of a parent glucoamylase.

3. The method according to claim 1, wherein the steamed flour-based product is selected from: steamed bread, steamed stuffed bun (bao zi), steamed twisted roll (hua juan), steamed roll (juan zi), steamed dumpling, braised noodles (men mian), steamed sponge cake (fa gao), steamed sponge rice cake (mi fa gao), spring festival cake (Nian Gao).

4. The method according to claim 1, wherein the dough or the paste is made from materials containing flour, preferably, wherein said flour is selected from the group consisting of wheat flour, corn flour, rye flour, barley flour, oat flour, rice flour, or sorghum flour, potato flour and combinations thereof.

5. The method according to claim 1, wherein the parent glucoamylase is obtained from a species of Penicillium, such as Penicillium oxicalum, Penicillium miczynskii, Penicillium russellii or Penicillium glabrum.

6. The method according to claim 1, wherein the mature variant comprises at least one amino acid modification in one or more (several) or all of the positions corresponding to positions 1, 2, 4, 6, 7, 11, 31, 34, 65, 79, 103, 132, 327, 445, 447, 481, 566, 568, 594 and 595 in SEQ ID NO: 1.

7. The method according to claim 1, wherein the at least one amino acid modification comprises a substitution in one or more or all of the positions corresponding to positions 1, 2, 4, 11, 65, 79 and 327 in SEQ ID NO: 1.

8. The method according to claim 1, wherein the at least one amino acid modification comprises a substitution in one or more or all of the positions corresponding to positions 1, 6, 7, 31, 34, 79, 103, 132, 445, 447, 481, 566, 568, 594 and 595 in SEQ ID NO: 1.

9. The method according to claim 1, wherein the at least one amino acid modification comprises a substitution in one or more or all of the positions corresponding to positions 1, 6, 7, 31, 34, 50, 79, 103, 132, 445, 447, 481, 484, 501, 539, 566, 568, 594 and 595 in SEQ ID NO: 1.

10. The method according to claim 1, wherein the mature thermostable variant has a thermostability improvement (Td) over its parent of at least 3° C.

11. The method according to claim 1, wherein the mature thermostable variant has a relative activity at 91° C. of at least 150.

12. The method according to claim 1, wherein the glucoamylase is a mature thermostable variant of a parent glucoamylase, wherein the variant is at least 71% identical to SEQ ID NO: 1, SEQ ID NO: 6, SEQ ID NO: 7 or SEQ ID NO: 8, e.g. at least 72%, e.g. at least 73%, e.g. at least 74%, e.g. at least 75%, e.g. at least 76%, e.g. at least 77%, e.g. at least 78%, e.g. at least 79%, e.g., at least 80%, e.g. at least 81%, e.g. at least 82%, e.g. at least 83%, e.g. at least 84%, e.g., at least 85%, e.g. at least 86%, e.g. at least 87%, e.g. at least 88%, e.g. at least 89%, e.g., at least 90%, e.g., at least 91%, e.g., at least 92%, e.g., at least 93%, e.g., at least 94%, e.g., at least 95%, e.g. at least 96%, e.g., at least 97%, e.g., at least 98%, e.g., at least 99%, e.g. at least 100% identical to SEQ ID NO: 1, SEQ ID NO: 6, SEQ ID NO: 7 or SEQ ID NO: 8.

13. The method according to claim 1, wherein, compared with the same conditions as other conditions but without adding the enzyme to the dough or the paste, after 0.1, 0,25, 0.5, 1, 2, 3, 4, 5, 6 or 7 days of storage at room temperature or low temperature of 4° C., the steamed flour-based product has reduced hardness and/or improved elasticity.

14. The method according to claim 1, wherein, compared with the same conditions as other conditions but without adding the enzyme to the dough or the paste, after 0.1, 0.25, 0.5, 1, 2, 3, 4, 5, 6 or 7 days of storage at room temperature or low temperature of 4° C., the steamed flour-based product has an improved sensory evaluation value.

15. The method according to claim 1, wherein, compared with the same conditions as other conditions but without adding the enzyme to the dough or the paste, after 0.1, 0.25, 0.5, 1, 2, 3, 4, 5, 6 or 7 days of storage at room temperature or low temperature of 4° C., re-steaming the steamed flour-based products, the re-steamed steamed flour-based product has an improved sensory evaluation.

16. The method according to claim 1, wherein the sensory evaluation is a comprehensive evaluation preferably.

17. A flour composition, a dough premix, or a paste premix, comprising flour and a thermostable glucoamylase, wherein the glucoamylase is mature thermostable variant of a parent glucoamylase as defined in claim 12.

18. A dough comprising flour and a thermostable glucoamylase, wherein the glucoamylase is a mature thermostable variant of a parent glucoamylase as defined in claim 12.

19. The method according to claim 7, wherein the at least one amino acid modification comprises a substitution in one or more or all of the positions corresponding to R1A, P2N, P4S, P11F, T65A, K79V and Q327F in SEQ ID NO: 1.

20. The method according to claim 9, wherein the at least one amino acid modification comprises a substitution in one or more or all of the positions corresponding to R1A, G6S, G7T, R31F, K34Y, E50R, K79V, S103N, A132P, D445N, V447S, S481P, T484P, E501A, N539P, D566T, T568V, Q594R and F595S in SEQ ID NO: 1.


SEQUENCE LISTING (10 sequences)

SEQ ID NO: 1 - protein, 595 aa, Penicillium oxalicum
RPDPKGGNLTPFIHKEGERSLQGILDNLGGRGKKTPGTAAGLFIASPNTENPNYYYTWTRDSALTAKCLIDLFEDSRAKFPIDRKYLETGIRDYVSSQAILQSVSNPSGTLKDGSGLGEPKFEIDLNPFSGAWGRPQRDGPALRATAMITYANYLISHGQKSDVSQVMWPIIANDLAYVGQYWNNTGFDLWEEVDGSSFFTIAVQHRALVEGSQLAKKLGKSCDACDSQPPQILCFLQSFWNGKYITSNINTQASRSGIDLDSVLGSIHTFDPEAACDDATFQPCSARALANHKVYVDSFRSIYKINAGLAEGSAANVGRYPEDVYQGGNPWYLATLGASELLYDALYQWDRLGKLEVSETSLSFFKDFDATVKIGSYSRNSKTYKKLTQSIKSYADGFIQLVQQYTPSNGSLAEQYDRNTAAPLSANDLTWSFASFLTATQRRDAVVPPSWGAKSANKVPTTCSASPVVGTYKAPTATFSSKTKCVPAKDIVPITFYLIENTYYGENVFMSGNITALGNWDAKKGFPLTANLYTQDQNLWFASVEFIPAGTPFEYKYYKVEPNGDITWEKGPNRVFVAPTGCPVQPHSNDVWQF

SEQ ID NO: 2 - protein, 595 aa, synthetic construct
  REGION 1..595: note = AMG NL
RPDPKGGNLTPFIHKEGERSLQGILDNLGGRGKKTPGTAAGLFIASPNTENPNYYYTWTRDSALTAKCLIDLFEDSRAVFPIDRKYLETGIRDYVSSQAILQSVSNPSGTLKDGSGLGEPKFEIDLNPFSGAWGRPQRDGPALRATAMITYANYLISHGQKSDVSQVMWPIIANDLAYVGQYWNNTGFDLWEEVDGSSFFTIAVQHRALVEGSQLAKKLGKSCDACDSQPPQILCFLQSFWNGKYITSNINTQASRSGIDLDSVLGSIHTFDPEAACDDATFQPCSARALANHKVYVDSFRSIYKINAGLAEGSAANVGRYPEDVYQGGNPWYLATLGASELLYDALYQWDRLGKLEVSETSLSFFKDFDATVKIGSYSRNSKTYKKLTQSIKSYADGFIQLVQQYTPSNGSLAEQYDRNTAAPLSANDLTWSFASFLTATQRRDAVVPPSWGAKSANKVPTTCSASPVVGTYKAPTATFSSKTKCVPAKDIVPITFYLIENTYYGENVFMSGNITALGNWDAKKGFPLTANLYTQDQNLWFASVEFIPAGTPFEYKYYKVEPNGDITWEKGPNRVFVAPTGCPVQPHSNDVWQF

SEQ ID NO: 3 - protein, 595 aa, synthetic construct
  REGION 1..595: note = AMG anPAV498
RNDSKGGNLTFFIHKEGERSLQGILDNLGGRGKKTPGTAAGLFIASPNTENPNYYYTWTRDSALAAKCLIDLFEDSRAVFPIDRKYLETGIRDYVSSQAILQSVSNPSGTLKDGSGLGEPKFEIDLNPFSGAWGRPQRDGPALRATAMITYANYLISHGQKSDVSQVMWPIIANDLAYVGQYWNNTGFDLWEEVDGSSFFTIAVQHRALVEGSQLAKKLGKSCDACDSQPPQILCFLQSFWNGKYITSNINTQASRSGIDLDSVLGSIHTFDPEAACDDATFQPCSARALANHKVYVDSFRSIYKINAGLAEGSAANVGRYPEDVYFGGNPWYLATLGASELLYDALYQWDRLGKLEVSETSLSFFKDFDATVKIGSYSRNSKTYKKLTQSIKSYADGFIQLVQQYTPSNGSLAEQYDRNTAAPLSANDLTWSFASFLTATQRRDAVVPPSWGAKSANKVPTTCSASPVVGTYKAPTATFSSKTKCVPAKDIVPITFYLIENTYYGENVFMSGNITALGNWDAKKGFPLTANLYTQDQNLWFASVEFIPAGTPFEYKYYKVEPNGDITWEKGPNRVFVAPTGCPVQPHSNDVWQF

SEQ ID NO: 4 - protein, 595 aa, synthetic construct
  REGION 1..595: note = AMG JPO-001
ANDSKGGNLTFFIHKEGERSLQGILDNLGGRGKKTPGTAAGLFIASPNTENPNYYYTWTRDSALAAKCLIDLFEDSRAVFPIDRKYLETGIRDYVSSQAILQSVSNPSGTLKDGSGLGEPKFEIDLNPFSGAWGRPQRDGPALRATAMITYANYLISHGQKSDVSQVMWPIIANDLAYVGQYWNNTGFDLWEEVDGSSFFTIAVQHRALVEGSQLAKKLGKSCDACDSQPPQILCFLQSFWNGKYITSNINTQASRSGIDLDSVLGSIHTFDPEAACDDATFQPCSARALANHKVYVDSFRSIYKINAGLAEGSAANVGRYPEDVYFGGNPWYLATLGASELLYDALYQWDRLGKLEVSETSLSFFKDFDATVKIGSYSRNSKTYKKLTQSIKSYADGFIQLVQQYTPSNGSLAEQYDRNTAAPLSANDLTWSFASFLTATQRRDAVVPPSWGAKSANKVPTTCSASPVVGTYKAPTATFSSKTKCVPAKDIVPITFYLIENTYYGENVFMSGNITALGNWDAKKGFPLTANLYTQDQNLWFASVEFIPAGTPFEYKYYKVEPNGDITWEKGPNRVFVAPTGCPVQPHSNDVWQF

SEQ ID NO: 5 - protein, 595 aa, synthetic construct
  REGION 1..595: note = AMG JPO-124
ANDSKSTNLTFFIHKEGERSLQGILDNLGGFGKYTPGTAAGLFIASPNTENPNYYYTWTRDSALAAKCLIDLFEDSRAVFPIDRKYLETGIRDYVSSQAILQNVSNPSGTLKDGSGLGEPKFEIDLNPFSGPWGRPQRDGPALRATAMITYANYLISHGQKSDVSQVMWPIIANDLAYVGQYWNNTGFDLWEEVDGSSFFTIAVQHRALVEGSQLAKKLGKSCDACDSQPPQILCFLQSFWNGKYITSNINTQASRSGIDLDSVLGSIHTFDPEAACDDATFQPCSARALANHKVYVDSFRSIYKINAGLAEGSAANVGRYPEDVYFGGNPWYLATLGASELLYDALYQWDRLGKLEVSETSLSFFKDFDATVKIGSYSRNSKTYKKLTQSIKSYADGFIQLVQQYTPSNGSLAEQYDRNTAAPLSANDLTWSFASFLTATQRRNASVPPSWGAKSANKVPTTCSASPVVGTYKAPTATFPSKTKCVPAKDIVPITFYLIENTYYGENVFMSGNITALGNWDAKKGFPLTANLYTQDQNLWFASVEFIPAGTPFEYKYYKVEPNGTIVWEKGPNRVFVAPTGCPVQPHSNDVWRS

SEQ ID NO: 6 - protein, 595 aa, synthetic construct
  REGION 1..595: note = AMG JPO-172
ANDSKSTNLTFFIHKEGERSLQGILDNLGGFGKYTPGTAAGLFIASPNTRNPNYYYTWTRDSALAAKCLIDLFEDSRAVFPIDRKYLETGIRDYVSSQAILQNVSNPSGTLKDGSGLGEPKFEIDLNPFSGPWGRPQRDGPALRATAMITYANYLISHGQKSDVSQVMWPIIANDLAYVGQYWNNTGFDLWEEVDGSSFFTIAVQHRALVEGSQLAKKLGKSCDACDSQPPQILCFLQSFWNGKYITSNINTQASRSGIDLDSVLGSIHTFDPEAACDDATFQPCSARALANHKVYVDSFRSIYKINAGLAEGSAANVGRYPEDVYFGGNPWYLATLGASELLYDALYQWDRLGKLEVSETSLSFFKDFDATVKIGSYSRNSKTYKKLTQSIKSYADGFIQLVQQYTPSNGSLAEQYDRNTAAPLSANDLTWSFASFLTATQRRNASVPPSWGAKSANKVPTTCSASPVVGTYKAPTATFPSKPKCVPAKDIVPITFYLIANTYYGENVFMSGNITALGNWDAKKGFPLTANLYTQDQPLWFASVEFIPAGTPFEYKYYKVEPNGTIVWEKGPNRVFVAPTGCPVQPHSNDVWRS

SEQ ID NO: 7 - protein, 593 aa, Penicillium miczynskii
NPTSKDAKMASYISKEGQRSIVGITENLGGKGSKTPGTAAGLFIASPNMANPNYYYTWTRDSALTFKCLIDLFETSDQDYISRKELETDIRNYVSSQAVLQNVSNPSGTLKDGSGLGEPKFEIDLNPFSGSWGRPQRDGPALRATAMITYADWLISHGSKSEAANIMWPIIANDLAYVGQYWNNTGFDLWEEVDGSSFFTLAVQHRSLVQGASLAKKLGKSCPACVSQAPQILCFLQSFWNGNYITANINLDTSRSGIDLNSILGSIHTFDPEASCDDSTFQPCSARALANHKVYVDAFRSIYKVNAGLSNGSAANVGRYPEDVYQGGNPWYLATLASAELLYDALYQWSKIGKLDVTKTSLAFFKDFDMAVKTGTYSAHSSTYKSLTSAIRTYADDFIGLVQDYTPSNGSLAEQYDRNTGIPLSANDLTWSYASFITAIQRRASVVPASWGEKSANVVPTICSASPVTGTYQAAPSVFPTTTGCVPATSIVPITFYLTETTFYGENVYMTGNISALGNWDTNNGFPLTANLYTDSDHLWFASVELVPAGTPFEYKYYKVEPNGTVIWENGENRVYVAPTGCPIQPSQTDVWR

SEQ ID NO: 8 - protein, 590 aa, Penicillium russellii
SKDGNLASYIAKEGQRSIVGITENLGGKGSKTPGTAAGLFIASPNMANPNYYYTWTRDSALTFKCLIDLFETSDQDYISRKELETDIRNYVSSQAVLQNVSNPSGTLKDGSGLGEPKFEIDLNPFSGSWGRPQRDGPALRATAMITYADWLVSHGQKSEATNIMWPIIANDLAYVGQYWNKTGFDLWEEVDGSSFYTLAVQHRALVQGASLAKKLGKSCTACVSQAPQILCFLQSFWNGNYITANINLDTSRSGIDLNSILGSIHTFDPEASCDDSTFQPCSARALANHKVYVDAFRSIYGVNAGLSNGTAANVGRYPEDVYQGGNPWYLATLAAAELLYDALYQWNQIGKLDVTKTSLAFFKDFDAAVKTGTYSAHSSAYRTLTSAIRTYADDFISLVQHYTPSNGSLAEQYDRDTGIPLSANDLTWSYASFITAIERRASVVPASWGEKSANVVPTTCSASPVTGTYVAATSVFPTTTGCVPATSIVPITFYLTESTFYGENVYMTGNISALGNWDTSSGFPLTANLYTDSDHLWFASVELVPAGTPFEYKYYKVEPNGTVIWENGENRVYVAPTGCPIQPSQTDIWR

SEQ ID NO: 9 - protein, 594 aa, Penicillium glabrum
NPFNSLDSFILKEGARSYQGIIDNLGNKGVKAPGTAAGLFVASPNTANPDYFYTWTRDSALTFKCLIDLFDGGSTEFGLKNSELETDIRNYVSSQAVLQNVSNPSGTLEDGTGLGEPKFEVDLNPFTGSWGRPQRDGPALRATALITYTNYLLSQGQKSEAVNIMWPIISNDLAYVGQYWNDTGFDLWEETDGSSFFTLAVQHRALVQGATLAQKLGKSCAACSSQAPQILCFLQSFWNGKYITANINLDTSRTGIDANTLLGSIHTFDPEAACDDSTFQPCSARALANHKVYVDAFRSIYKINSGIAEGSPANVGRYPEDVYQGGNPWYLTTLASAELLYDALYQWNKIGGLDVTETSLAFFKDFHSSVKTGSYSAHSQTYKTLTSAIRTYADGFVGLVQKYTPANGSLAEQYNRNTSVPLSANDLTWSFASFLTAIQRRESIVPGSWGEKSANTVPTTCSASPVTGTYKAATSTFPTSTAGCVPATDVVPITFYLIENTYYGENVYMTGNISALGNWDTSDGLALDAGLYTETDNLWFGTLELVTAGTPFEYKYYKIEPNGTVTIWESGDNRVSVVPTGCPIQPSLHDVWRS

SEQ ID NO: 10 - protein, 591 aa, Rasamsonia emersonii
ATGSLDSFLATETPIALQGVLNNIGPNGADVAGASAGIVVASPSRSDPNYFYSWTRDAALTAKYLVDAFIAGNKDLEQTIQQYISAQAKVQTISNPSGDLSTGGLGEPKFNVNETAFTGPWGRPQRDGPALRATALIAYANYLIDNGEASTADEIIWPIVQNDLSYITQYWNSSTFDLWEEVEGSSFFTTAVQHRALVEGNALATRLNHTCSNCVSQAPQVLCFLQSYWTGSYVLANFGGSGRSGKDVNSILGSIHTFDPAGGCDDSTFQPCSARALANHKVVTDSFRSIYAINSGIAEGSAVAVGRYPEDVYQGGNPWYLATAAAAEQLYDAIYQWKKIGSISITDVSLPFFQDIYPSAAVGTYNSGSTTFNDIISAVQTYGDGYLSIVEKYTPSDGSLTEQFSRTDGTPLSASALTWSYASLLTASARRQSVVPASWGESSASSVPAVCSATSATGPYSTATNTVWPSSGSGSSTTTSSAPCTTPTSVAVTFDEIVSTSYGETIYLAGSIPELGNWSTASAIPLRADAYTNSNPLWYVTVNLPPGTSFEYKFFKNQTDGTIVWEDDPNRSYTVPAYCGQTTAILDDSWQ